(19) United States
(12) Patent Application Publication
(10) Pub. No.: US 2004/0267131 A1
(43) Pub. Date: Dec. 30, 2004

(54) Method and product for depicton of the heart

(76) Inventor: Mia Norman Andersen, Viby (DK)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/495,657

(22) PCT Filed: Nov. 19, 2002
(86) PCT No.: PCT/DK02/00774

(30) Foreign Application Priority Data
Nov. 20, 2001 (DK) PA 2001 01729

(51) Int. Cl. A61B 8/14

(52) U.S. Cl. 600/459

(57) ABSTRACT

The invention relates to a method for ultrasound-based three-dimensional imaging of a heart in connection with open heart surgery. The method comprises using a liquid as an interface medium between an ultrasound transducer and a heart after opening of the breastbone prior to a surgical intervention in the heart. The invention moreover comprises a liquid reservoir product to be placed on the surface of the breast of patients who have had their breast part opened prior to an open heart operation. The liquid reservoir creates a liquid-tight volume between the heart and an ultrasound transducer which may be filled with a liquid, such as an isotonic salt water solution, for the purpose of filling the gap between the transducer and the heart with the liquid, thereby optimizing the image quality from the ultrasound scanning.

The invention relates to a reservoir for a liquid, such as isotonic salt water, supplied to the heart region in open heart surgery in animals or humans, said liquid being adapted to receive an ultrasonic scanner.

In connection with open heart surgery it is of great clinical importance that the surgeon can have a three-dimensional image of the heart immediately before the intervention in the heart. The better knowledge the surgeon has of the state of the heart immediately before the intervention in the heart, the better the surgical intervention can be planned and optimized, which, in turn, can contribute to improving the result of the surgical intervention.

The organ imaging techniques which are used today in connection with the diagnosis of cardiac diseases and the planning of surgical interventions, e.g. comprise MR (magnetic resonance), CT (X-ray tomography) as well as ultrasound technology.

With modern technology, MR and CT scannings are normally performed in large scanners which are located in special examination rooms, which cannot also be used for surgery. Therefore, it is not possible to use these technologies in connection with e.g. open heart surgery.

Ultrasound scanning systems are physically smaller as well as easily transportable and therefore lend themselves for use in operation rooms.

U.S. Pat. No. 5,964,707 discloses an apparatus, in which an ultrasound transducer having a two-dimensional scanner head is moved linearly so as to produce a three-dimensional reconstruction of the organ under examination as a result of the overall scanning. It is mentioned in the application that the apparatus may be used in connection with surgery, but it is not explained which types of surgery are considered, or how the apparatus is to be used in such cases during surgery.

It is likewise known from the literature to ultrasound-scan in 3 dimensions with fixed ultrasound probes.

Ultrasound scanning of the heart in animals or humans is also known, such scannings being performed by placing the ultrasound transducer on the thoracic cage of the individual to be measured. This technique, however, does not give a complete image of the heart, as the ordinary ultrasound technique cannot image organs or tissue behind ribs, breastbones or bones.

To avoid the disadvantageous shadow effect that occurs when the ultrasound signals are to pass ribs and/or breastbones, it is known to try to scan with the ultrasound probe positioned externally on the skin of the individual between two ribs.

This method, however, has the drawback that the visual field in which the transducer can scan the heart without the ultrasound signals hitting the heart, is rather limited, which means in practice that it is normally not possible to image the entire heart with this method.

It is evident that the larger the heart is relative to the spacing between two ribs between which scanning is performed, the smaller part of the heart can subsequently be imaged three-dimensionally without the image being adversely affected by the ribs.

Many cardiac diseases result in the phenomenon that the heart is enlarged as the disease develops, which therefore means that three-dimensional ultrasound scanning from many of these patients is uninteresting in clinical practice, as only a small part of the heart can be imaged with the known technique.

In addition to the disadvantages mentioned in connection with the prior art, it is a drawback that methods or devices for use in three-dimensional ultrasound scanning in connection with open heart surgery are not known.

Accordingly, an object of the invention is to improve the known apparatus for three-dimensional imaging of the heart in humans or animals such that it may be used in connection with open heart surgery.

The object of the invention is achieved by a reservoir of the type defined in the introductory portion of claim 1, which is characterized in that that the reservoir is made of a formable plastic material, such as a polymer, having a basic shape as a cylinder section comprising a wall having a lower side which forms a boundary between a patient's breast and the lower side.

The reservoir provides the advantage that a closed boundary is arranged around the opening in the thoracic cage which is provided by cutting the breastbone as an initial step of the open heart surgical intervention, said closed boundary serving as side walls in a basin which prevent liquid supplied to the heart region from running away.

Expedient embodiments of the reservoir are defined in claims 2-5.

The invention will now be explained more fully with reference to the drawings, in which

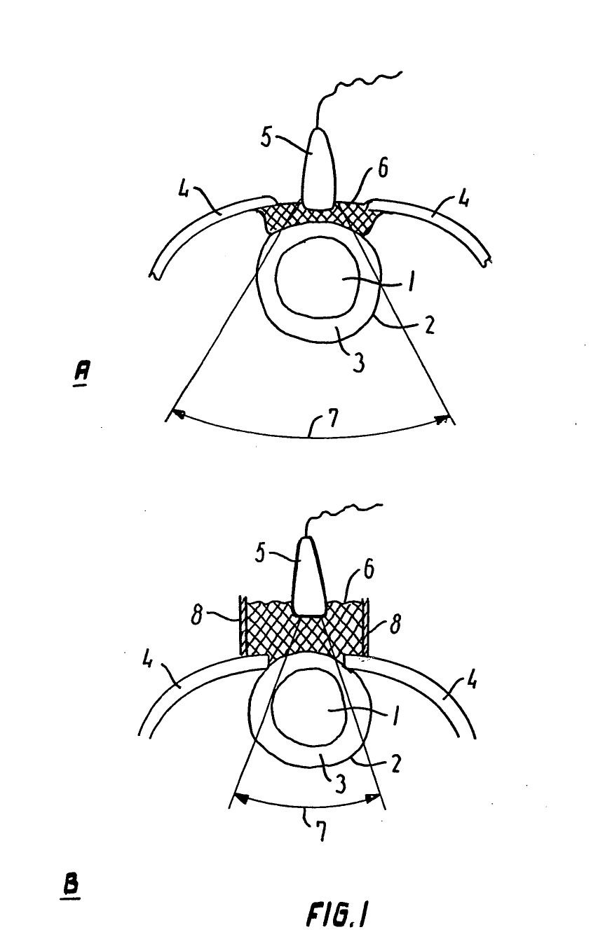

FIG. 1A, in cross-section through the heart region of an animal or a human, shows an ultrasound transducer which measures on a heart after opening of the breastbone where a depression between the breast surface and the heart is filled with liquid which fills the gap between transducer and heart,

FIG. 1B, in cross-section through the heart region, shows an ultrasound scanning of a heart after opening of the breastbone, where liquid between the ultrasound transducer and the heart is prevented from running away from the heart region by a liquid reservoir in the form of coherent liquid-tight side faces placed on the surface of the breast,

FIG. 2A shows a patient seen from the side and placed on a surgical bed with a liquid reservoir product arranged on the breast around the opening in the breastbone which has been made prior to an open heart surgical intervention,

FIG. 2B shows the same subject as is described with reference to FIG. 2A, but the patient is seen from above in FIG. 2B,

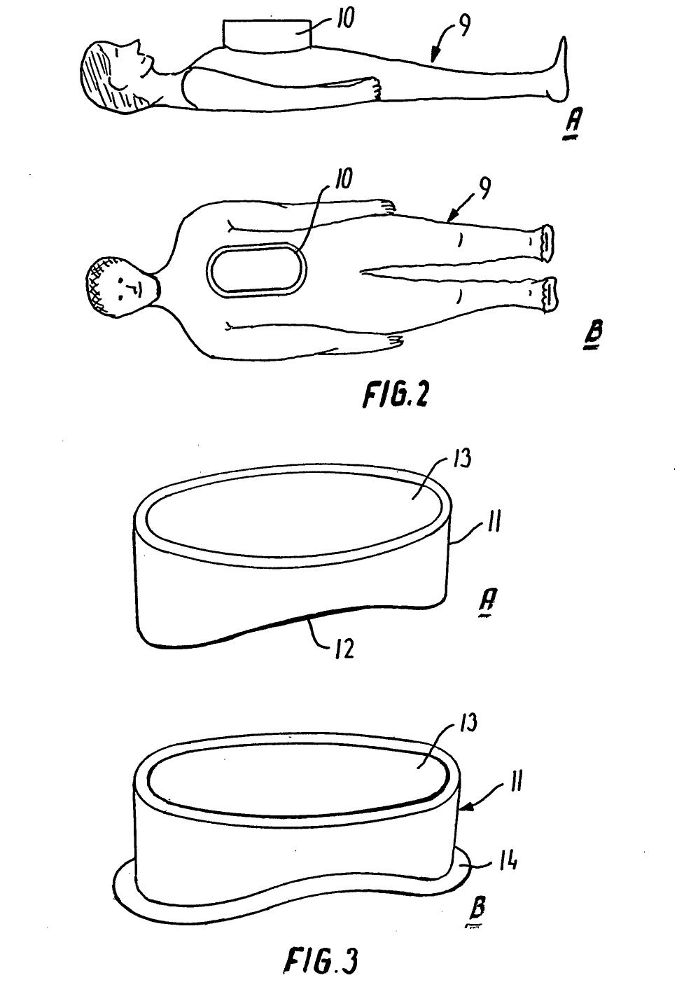

FIGS. 3A and 3B show two examples of the arrangement of liquid reservoirs for open heart surgery in accordance with the present invention,

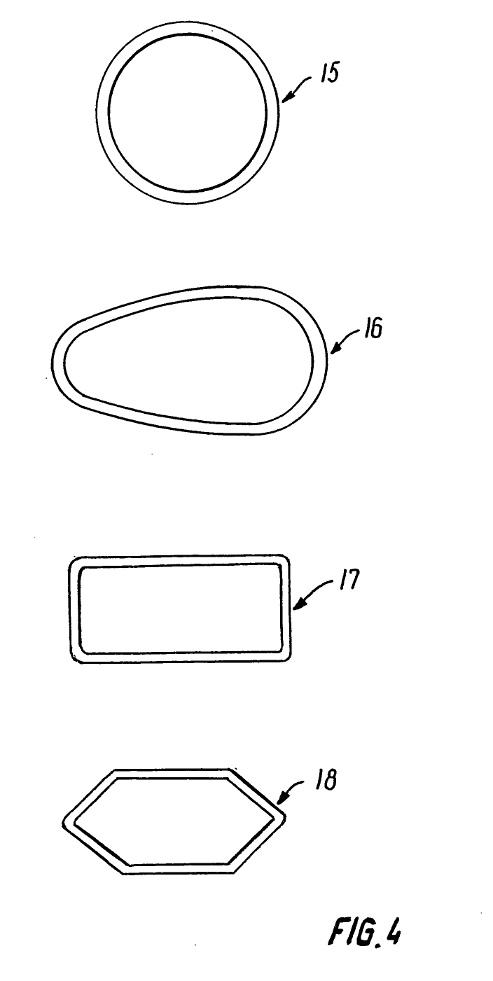

FIG. 4 shows three examples seen from above of the arrangement of liquid reservoirs for open heart surgery in accordance with the present invention.

FIGS. 1A and 1B show, in cross-section through the heart region, basic sketches of an ultrasound examination performed on an individual who has had his breastbone, which joins the ribs 4 on the right side and the left side, opened to expose the heart 1.

FIGS. 1A and 1B show an ultrasound transducer 5 positioned in the opening of a breastbone of an animal or a human, from which the transducer within its visual field 7 can see the entire heart 1 which is enclosed by pericardial liquid 3 enclosed by a pericardium 2.

Liquid 6, typically a sterile isotonic salt water solution, is present between a transducer 5 and a pericardium 2 in the shown case. If the pericardium is broken, the isotonic salt water will directly fill the gap between the transducer and the heart.

FIG. 1A shows a situation where there is a sufficient natural depression between the surface 6 of a breast defined by ribs 4 and pericardium 2 to allow the depression to be filled with liquid for filling the gap between the transducer 5 and the pericardium 2.

This ensures that liquid is present between transducer and heart, and that there are therefore no air gaps which may impair the image quality of the three-dimensional ultrasound scanning of the heart.

A method like the one outlined in FIG. 1A will make it possible to perform a three-dimensional ultrasound scanning of the heart prior to the surgical intervention in the heart.

The liquid which is filled into the depression in the heart region after the opening of the breastbone, is important only to the actual ultrasound examination, and therefore it is removed again before the surgical intervention in the heart is commenced.

In practice, the liquid may be poured directly into the depression from a container and be discharged again by suction by means of e.g. a liquid suction pump.

When a breastbone is cut as an initial step of an open heart surgical intervention, the pressure conditions in the body, however, will frequently cause the heart behind the breastbone to be pressed up toward the opening in the breast, which is shown schematically in FIG. 1B.

In FIG. 1B, a heart 1 has been pressed up between an opening which is defined by ribs 4, so that a pericardium 2 balloons from the opening in the breast.

When, as shown in FIG. 1B, a heart is pressed up toward an opening in a breastbone, there will no natural depression into which liquid may be poured for the purpose of filing the gap between an ultrasound transducer 5 and a heart 1, or a pericardium 2 in the event that the pericardium is unbroken.

By placing a liquid reservoir 8 on top of the breast of an individual to receive heart surgery it is possible artificially to create a cavity 6 into which the desired liquid may be filled.

The liquid reservoir 8 thus forms the sides of a liquid-tight basin, where a heart 1, or a pericardium 2 in the case shown in FIG. 1B, and other body parts, such as ribs 4, form the bottom.

The product 8 thus ensures that the gap between a transducer 5 and a heart 1 may be filled with liquid, even though the heart 1 or the pericardium 2 is pressed entirely or partly up and fills the gap produced by the opening of the breastbone.

As will appear from FIG. 1B, it will frequently be necessary to establish a certain distance between the transducer 5 and the heart 1 for the reason that the visual field 7 from the transducer has a limit, which quite simply requires that the transducer is kept at a minimum distance from the heart in order for the entire volume of the heart to be accommodated within the visual field 7.

FIG. 2 shows a heart patient 9 placed on a surgery bed, where 2A shows the patient seen from the side, while 2B shows the patient seen from above. An example of a liquid reservoir product 10, which forms part of the invention, is placed on the patient on the breast around the opening in the breastbone, which has been made as an initial step in an open heart surgical intervention.

As will appear from FIG. 2, the product 10 forms the sides of a basin where the bottom is constituted by the open heart region of the patient.

The liquid reservoir product 10 is made of a liquid-tight material, such as a polymer, and the material must moreover be plastic so that it may easily be formed to sealingly engage the breast surface of the patient.

To ensure that no liquid can run out from the contact face between the patient's breast surface and the bottom of the liquid reservoir 10, it may be expedient to fill the contact face with a liquid-tight soft and sticky filler or gluing material.

FIG. 3 shows two examples of preferred embodiments of liquid reservoir products 11 made in accordance with the present invention.

The liquid reservoir consists of an unbroken and coherent face 11 having a lower side 12 which is placed on the patient's breast around the opening in the breastbone.

It is evident that for the liquid reservoir product to operate according to the purpose, it must have a circumference which is sufficiently large for the entire opening in the breast to be enclosed by the liquid reservoir.

The lower end face 12, which is in contact with the patient, may advantageously be formed so that the surface has the same contour as the surface of the patient on which the liquid reservoir product is to be placed.

FIG. 3A shows an example of a liquid reservoir having plane side faces.

Frequently, however, it will be advantageous to provide the liquid reservoir with a collar 14 on the end which is placed against the patient's breast surface. This facilitates application of a liquid tight adhesive material, which prevents liquid from escaping from the contact face between the breast and the liquid reservoir. When the liquid reservoir is placed on the patient's breast surface, the liquid to fill the gap between the heart and the ultrasound transducer in the scanning may be filled into the liquid reservoir volume 13.

In connection with open heart surgery, a sterile piece of plastics will frequently be placed across the patient's breast surface prior to the opening of the breast region. The plastics material inter alia has a bacteriologically inhibiting effect.

When such a plastics material is used, it will of course also be placed between the patient's breast surface and the liquid reservoir. Since the plastics material is liquid-tight, it has no adverse impact on either the method part or the product part of the invention, however.

If the above-mentioned bacterially inhibiting plastics material is used for open heart surgery, the plastics material in the present description should be regarded as the surface of the breast.

Liquid reservoirs made in accordance with the present invention may assume arbitrary shapes, some examples of which are shown in top view in FIG. 4.

A liquid reservoir having an almost circular cross-section of a plane in parallel with the surface of the breast on which the reservoir is placed, is shown in FIGS. 4-15.

FIGS. 4-16 show, in the same cross-section as 15, a shape which may be described as being composed of two ellipse-like figures.

FIGS. 4-17 show a shape inspired by a rectangle.

FIGS. 4-18 show a shape produced by addition of several partially linear subelements.

The shown FIGS. 4-15 to 4-18 are just examples of arrangements of liquid reservoirs seen in cross-section in parallel with the patient's breast surface. Thus, the figures do not restrict the scope of the present invention, which covers all arrangements of liquid reservoirs made or used for the purpose of enabling ultrasound scannings of the heart in connection with open heart surgery.

The invention moreover comprises liquid reservoir products provided with means for fixing the ultrasound transducer, such as mechanical brackets or other clamping devices.

The ultrasound examinations associated with the invention also comprise two-dimensional imaging in addition to the mentioned three-dimensional imaging. In connection with the ultrasound examinations of the heart in accordance with the invention, the ultrasound measurements may be used independently, or the ultrasound measurements may be temporally synchronized with the electrocardiographic (ECG) signal from the individual being measured.

Although the invention has been explained in connection with a method and a product for imaging the heart, nothing prevents the principles of the invention from being applied in other connections within the scope of the invention defined in the claims.

This might e.g. be in connection with the drafting of utility model applications.

1-5. (Cancel).

6. A reservoir for a liquid such as isotonic salt water, supplied to the heart region in open heart surgery in animals or humans, said reservoir being made of formable plastics material, such as a polymer, having a basic share as a cylinder section comprising a wall which may be placed around the opening in a thoracic cage, so that the inner side of the shaped cylinder wall forms a boundary within which liquid may be supplied to the heart region.

7. A reservoir according to claim 6, wherein the reservoir is provided with means, such as mechanical brackets, for fixing an ultrasound transducer.

8. A reservoir according to claim 7, wherein an ultrasound transducer is positioned above the reservoir at a distance from the heart of 1 and 100 mm.

9. A reservoir according to claim 8, wherein the reservoir is filled with the liquid prior to an ultrasonic measurement and removed after an ultrasonic measurement.

10. A method for three-dimensional imaging of the heart of an animal or a human under open heart surgery or surgical opening of the breastbone, wherein the measurement is performed by an ultrasound transducer, said ultrasound transducer being placed in a position above a liquid reservoir.

11. A method according to claim 10, wherein the ultrasound transducer is positioned at a distance from the heart of between 1 and 100 mm, the gap between the ultrasound transducer and the heart being filled with liquid.

12. A method according to claim 11, wherein the liquid is an isotonic salt water.

13. A method for three-dimensional imaging of the heart of an animal or human according to claim 10, wherein liquid, such as isotonic salt water, is supplied to the heart region prior to the ultrasound measurement and is removed again after the measurement has been accomplished.
